(19) United States
(12) Patent Application Publication
(10) Pub. No.: US 2024/0099865 A1
(43) Pub. Date: Mar. 28, 2024

(54) IMPLANTABLE CONDUIT WITH SELF-SEALING FENESTRATIONS, AND ASSOCIATED SYSTEMS AND METHODS

(71) Applicant: W. L. Gore & Associates, Inc., Newark, DE (US)

(72) Inventors: Kehinde A. Majolagbe, Flagstaff, AZ (US); James D. Silverman, Flagstaff, AZ (US)

(21) Appl. No.: 18/276,149

(22) PCT Filed: Feb. 5, 2021
(86) PCT No.: PCT/US2021/016811

§ 371 Date: Aug. 7, 2023

(51) Int. Cl. A61F 2/852; A61F 2/06; A61F 2/88

(52) U.S. Cl. CPC A61F 2/852; A61F 2/06; A61F 2/88; A61F 2002/7806

(57) ABSTRACT

Examples of an implantable conduit that is tubular in shape and has an inner lumen and an outer surface, the implantable conduit are disclosed. The implantable conduit includes a plurality of overlap elements including a first overlap element and a second overlap element positioned adjacent the first overlap element to define an adjacent pair of the plurality of overlap elements. The first overlap element overlaps onto the second overlap element to define an overlapping portion between an inward facing surface of the first overlap element and an outward facing surface of the second overlap element. The implantable conduit also includes a plurality of attachment features coupling the first overlap element to the second overlap element. The inward facing surface of the first overlap element and the outward facing surface of the second overlap element are separable at the overlapping portion by an external force to open an aperture in the overlapping portion between at least two of the plurality of attachment features such that the aperture defines a path between the inner lumen and outer surface of the implantable conduit. The path is sealed absent the external force.

### CROSS-REFERENCE TO RELATED APPLICATION

This application is a national phase application of PCT Application No. PCT/US2021/016811, internationally filed on Feb. 5, 2021, which is herein incorporated by reference in its entirety for all purposes.

### FIELD

This document relates to implantable intraluminal medical devices. For example, this document relates to implantable conduits that can be implanted in bodily cavities, organs, and vessels.

### BACKGROUND

In numerous locations of the human anatomy, a primary bodily conduit (e.g., primary blood vessel) is connected with one or more secondary bodily conduits (e.g., branch or peripheral vessels) that branch off from the primary bodily conduit. In some cases the secondary bodily conduits convey fluid into the primary bodily conduit (e.g., venous conduits), while in other cases the secondary bodily conduits convey fluid away from the primary bodily conduit (e.g., arterial conduits).

The human vasculature includes many examples of primary bodily conduits that have secondary branches. One example of a primary bodily conduit is the aorta. And, in the aortic arch region, three major arteries branch off from the aorta. Those three arteries are the brachiocephalic artery, the left common carotid artery, and the left subclavian artery, and they conduct fluid away from the aorta.

Bodily conduits and/or circulatory (or other bodily) systems with which they are associated may suffer from various diseases, defects and conditions, or may otherwise benefit from augmentation using one or more implantable conduits, including grafts, stent-grafts, filters, anastomosis devices, prosthetic valves, and others. Generally, such implantable conduits have tubular forms configured to convey fluid in the body.

### SUMMARY

Disclosed herein are examples of an implantable conduit that is tubular in shape and has an inner lumen and an outer surface, the implantable conduit. According to one example, (“Example 1”), the implantable conduit includes a plurality of overlap elements including a first overlap element and a second overlap element positioned adjacent the first overlap element to define an adjacent pair of the plurality of overlap elements. The first overlap element overlaps onto the second overlap element to define an overlapping portion between an inward facing surface of the first overlap element and an outward facing surface of the second overlap element. The implantable conduit also includes a plurality of attachment features coupling the first overlap element to the second overlap element. The inward facing surface of the first overlap element and the outward facing surface of the second overlap element are separable at the overlapping portion by an external force to open an aperture in the overlapping portion between at least two of the plurality of attachment features such that the aperture defines a path between the inner lumen and outer surface of the implantable conduit. The path is sealed absent the external force.

According to another example (“Example 2”) further to Example 1, the plurality of overlap elements are arranged along a length of the implantable conduit and the overlapping portion extends in a circumferential direction.

According to another example (“Example 3”) further to Example 1 or 2, the plurality of overlap elements define a plurality of adjacent pairs of overlap elements and a plurality of overlapping portions. Each of the adjacent pairs of overlap elements is separable at a corresponding one of the plurality of overlapping portions to open an aperture that defines a path between the inner lumen and the outer surface of the implantable conduit.

According to another example (“Example 4”) further to any preceding Example, the plurality of attachment features are elongate strips of material extending in a longitudinal direction along a length of the implantable conduit to couple the first overlap element to the second overlap element.

According to another example (“Example 5”) further to any preceding Example, the implantable conduit is configured to close the aperture in the overlapping portion upon removal of the external force separating the overlap elements at the overlapping portion to seal the path between the inner lumen and outer surface of the implantable conduit.

According to another example (“Example 6”) further to any preceding Example, the attachment features include adhesive material.

According to another example (“Example 7”) further to any preceding Example, the attachment features are positioned at circumferentially staggered positions from one another about a circumference of the implantable conduit.

According to another example (“Example 8”) further to any preceding Example, the implantable conduit further includes a secondary device structure received through the aperture.

According to another example (“Example 9”) further to any preceding Example, the implantable conduit defines a varying diameter.

According to another example (“Example 10”) further to any preceding Example, the implantable conduit further includes a plurality of stent elements coupled to the plurality of overlap elements.

According to another example (“Example 11”) further to any one of Examples 1 to 10, the plurality of overlap elements are defined by a continuous, helical length of material overlapped edgewise onto itself, or the plurality of overlap elements include discrete, adjacent circumferential rings of material that are overlapped in an edgewise manner.

According to another example (“Example 12”) further to any one of Examples 1 to 10, the plurality of overlap elements are defined by a tubular body having a pleated configuration to define a plurality of pleats. The plurality of attachment features couple the overlap elements in the pleated configuration.

According to another example (“Example 13”) further to Example 12, the pleats are circumferentially oriented, and optionally, the aperture is formed in one of the pleats.

Also disclosed herein are examples of an implantable conduit which, according to one example (“Example 14”), includes a tube of flexible material having a length, a first end, and a second end. The tube is formed with multiple pleats along its length. The implantable conduit also includes a stiffening element reinforcing the pleats so as to resist the pleats from unfolding upon application of tension along the length of the tube. The tube is configured to be fenestrated within a pleat to provide a side portal along the length of the tube to allow for the introduction of a side branch tube through the portal. The pleat is configured to overlap and resist leakage around the side branch tube.

According to another example (“Example 15”) further to Example 14, the tube includes a radiopaque marker to indicate a point of fenestration.

According to another example (“Example 16”) further to Example 14, the stiffening element comprises a stent element.

According to another example (“Example 17”) further to Example 14, the stiffening element comprises a tape.

Also disclosed herein are examples of an implantable conduit which, according to one example (“Example 18”), includes a tube of flexible material having a length, a first end, and a second end. The tube is formed with multiple pleats along its length. The implantable conduit also includes a longitudinal element attached along the length to resist the pleats from unfolding upon application of tension along the length of the tube. The tube is configured to be fenestrated within a pleat to provide a side portal along the length of the tube to allow for the introduction of a side branch tube through the portal. The pleat is configured to overlap and resist leakage around the side branch tube.

According to another example (“Example 19”) further to Example 18, the longitudinal element comprises at least one spine.

According to another example (“Example 20”) further to Example 18 or 19, the longitudinal element comprises at least one tape.

Also disclosed herein are examples of a method of making an implantable conduit. According to one example, (“Example 21”), the method includes forming an implantable conduit by winding a sheet of membrane. The implantable conduit includes a plurality of overlap elements arranged in rows, and each overlap element overlaps an adjacent row of overlap element. The method also includes attaching a plurality of attachment features to the plurality of overlap elements. The plurality of attachment features are arranged essentially perpendicularly to the overlap elements to maintain the plurality of overlap elements in orientation with each other, and the implantable conduit is configured to be fenestrated to form an aperture at one or more locations along the implantable conduit by selectively separating the overlap elements from each other intermediate to the attachment features.

According to another example, (“Example 22”), the method of making an implantable conduit includes forming an aperture at one or more locations of an implantable conduit comprising a sheet of membrane, and folding the implantable conduit to form a plurality of pleats. The aperture is at the one or more locations within the pleats, and attaching a plurality of attachment features to the folded implantable conduit. The attachment features are arranged essentially perpendicularly to the pleats to maintain the pleats against a surface of the implantable conduit to keep the aperture closed, and the aperture at the one or more locations within the pleats is configured to be opened in response to selectively and at least partially unpleating the pleats from the surface of the implantable conduit intermediate to the attachment features.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a side view of an example of an implantable conduit, according to some embodiments;

FIG. 2 shows a side view of an implantable conduit with longitudinal attachment features, according to some embodiments;

FIG. 3 shows a close-up view of a portion of an outer surface of an implantable conduit, according to some embodiments;

FIG. 4 shows a side view of another implantable conduit, according to some embodiments;

FIG. 5 shows an unassembled view of the implantable conduit of FIG. 4, according to some embodiments;

FIG. 6 shows an individual overlap element of an implantable conduit, according to some embodiments;

FIG. 7 shows another individual overlap element of the implantable conduit of FIG. 5, according to some embodiments;

FIG. 8 shows an example of a segment of material used to form an implantable conduit according to some embodiments;

FIG. 9 shows an example of the segment of material shown in FIG. 6 partially wrapped into a tubular shape (with a manufacturing aid, such as a wrapping mandrel not being shown), according to some embodiments;

FIG. 10 is a side view of an implantable conduit including one or more stent elements, according to some embodiments;

FIG. 11 is a side view of an implantable conduit with a tapered configuration, according to some embodiments;

FIG. 12 shows a side view of another implantable conduit with longitudinal attachment features, according to some embodiments;

FIG. 13 shows a side view of another implantable conduit with stent elements, according to some embodiments;

FIG. 14 shows a side view of a segment of material (e.g., a tubular segment of material) with a plurality of apertures for forming an implantable conduit, according to some embodiments;

FIG. 15 shows a side view of an implantable conduit formed with the segment of material of FIG. 14, according to some embodiments;

FIG. 16 shows a longitudinal section of a portion of the implantable conduit of FIG. 15 with a plurality of apertures shown in a closed configuration, according to some embodiments;

FIG. 17 shows a longitudinal section of a portion of the implantable conduit of FIG. 15 with a plurality of apertures shown in an open configuration, according to some embodiments;

FIG. 18 shows a side view of a segment of material (e.g., a tubular segment of material) with a plurality of apertures for forming an implantable conduit, according to some embodiments; and

FIG. 19 shows a longitudinal section of a portion of the tube of FIG. 18 with a plurality of pleats formed upon folding, according to some embodiments.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” and similar terminology can be understood to mean plus or minus 20% of the stated value.

### Description of Various Embodiments

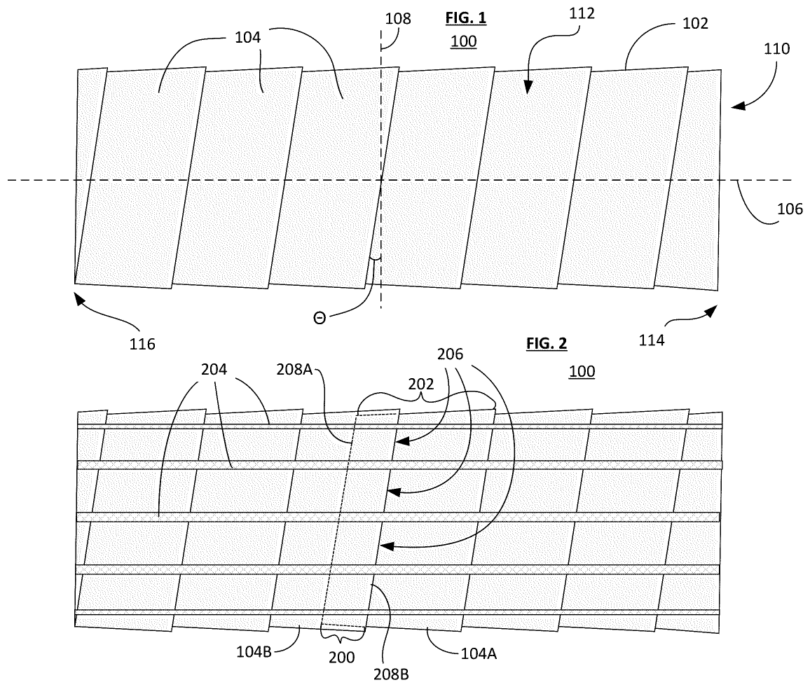

FIGS. 1 and 2 show an implantable conduit 100 (e.g., graft, stent graft, anastomosis device, prosthetic valve, or other implantable conduit) according to some embodiments. In various examples, the implantable conduit 100 is implantable within vasculature of a patient during a medical procedure (e.g., surgically or using percutaneous approaches). The implantable conduit 100 includes a cover or covering 102, which may also be described as a wall 102 or body 102, formed by a plurality of layer elements 104, also described as overlap elements 104.

In some examples, the implantable conduit 100, and in particular, the body 102, is formed of one or more layers of biocompatible material, such as one or more layers of biocompatible, polymeric material. Some examples of suitable biocompatible materials include least one of polyether ether ketone (PEEK), expanded polytetrafluoroethylene (ePTFE), fluorinated ethylene propylene (FEP), copolymers of tetrafluoroethylene (TFE) and perfluoromethyl vinyl ether (PMVE), also described as TFE-PMVE copolymers, urethanes, polyesters, thermoplastics, thermosets, nylon, polyethylene, expanded polyethylene, ultra-high-molecular-weight polyethylene (UHMWPE), or any of a variety of other biocompatible materials. In some examples, suitable materials may include composite membranes, such as an expanded polytetrafluoroethylene (ePTFE) based composite membrane or fibrillated polypropylene based composite membrane as well as any composite membrane based on one or more of the previously mentioned biocompatible materials. In addition to membrane materials, the implantable conduit 100 can be made from various fabrics that are woven, braided or knitted from various types of natural or synthetic fibers, including but not limited to PET fibers, polyester fibers, polyamide fibers, PTFE fibers, ePTFE fibers, and others.

As shown, the overlap elements 104 are arranged in rows such that each of the overlap elements 104 overlap (e.g., defines an overlapping edge) to define overlapping portions 200 with neighboring or adjacent ones of the overlap elements 104. In various examples the overlap elements 104 are arranged essentially axially, or in line with a central longitudinal axis 108 of the implantable conduit 100. The implantable conduit 100 also has a plurality of attachment features 204 at desired intervals (e.g., at desired intervals around the circumference) such that the plurality of overlap elements 104 are arranged along the length of the implantable conduit 100 and one or more of the overlapping portions 200 (e.g., all of them) extend in a circumferential direction. For reference, a “circumferential direction” need not be purely circumferential, or extending in a transverse plane to the longitudinal axis, but may also include a helical direction. The attachment features 204 each correspond to an area where the adjacent ones of the overlap elements 104 are bonded, adhered, secured, fastened, or otherwise coupled to one another. In various examples, the attachment features 204 are located along the overlapping portions 200 defined by the overlap elements 104.

There may be a consistent amount of overlap between the overlap elements 104 or the amount of overlap may vary along the length of the implantable conduit 100. In some examples, the overlap between overlap elements 104 is any of the following values, or any range between any of the following values: 10%, 20%, 30%, 40%, 50%, 60%, 70%, 80% or more of the individual lengths of the overlap elements 104. In different terms, in some examples the overlapping portions 200 between neighboring overlap elements 104 may be from 10% to 20%, 20% to 30%, 30% to 40%, 40% to 50%, 50% to 60%, 60% to 70%, 70% to 80%, or any other suitable value or range therebetween, of a width 202 of the overlap element 104 (as measured in the lengthwise direction of the implantable conduit 100).

As shown, the implantable conduit 100 has a generally cylindrical or tubular shape with an inner lumen 110 and an outer surface 112 (e.g., the outer surface of the wall or body 102). The implantable conduit 100 may have a generally constant diameter along all or one or more portions of the length of the implantable conduit, or a varying diameter along all or one or more portions of the length of the implantable conduit (e.g., one or more portions that have an overall tapering or stepped in diameter) as desired. The outer profile of the implantable conduit 100 may be irregular (e.g., ridged or undulating) as a result of the overlapping arrangement of the overlap elements 104. In this context, the “outer diameter” of the implantable conduit is meant to indicate the average diameter defined by the overlap elements 104, which in FIG. 1 is generally constant for the portion of the implantable conduit 100 shown. The implantable conduit 100 has a first end 114 and a second end 116 that define a length therebetween.

Although the implantable conduit 100 is depicted with overlap elements 104 along the entire length shown, one or more portions of the implantable conduit 100 may not include overlap elements (e.g., being formed as a single, integral unitary length of material). Additionally, though the overlap elements 104 are shown with a relatively constant size and shape, the overlap elements 104 may vary in size and shape, including the amount of overlap defined by adjacent overlap elements 104.

In some examples, one or more (e.g., all) of the overlap elements 104 are defined by a continuous, helical length of material overlapped edgewise onto itself at a desired pitch. In such examples, each of the overlap elements 104 may be considered a single turn, or revolution of the material forming the overlap elements 104. In other examples, one or more (e.g., all) of the overlap elements 104 include discrete, adjacent circumferential rings of material that are overlapped in an edgewise manner, where individual ones of the circumferential rings forming the overlap elements 104.

In examples including overlap elements 104 formed by a continuous, helical length of material, because the implantable conduit 100 is made the continuous length of the body 102, each of the overlap elements 104 is continuously connected to neighboring ones of the overlap elements 104. The width 202 of the overlapping portions 200 (the amount of overlap in the lengthwise direction of the implantable conduit 100) can be adjusted by varying a wrapping angle Θ of the covering 102 with respect to an axis 108 perpendicular to the implantable conduit 100 or transverse to a center of the implantable conduit 100.

As previously referenced, the attachment features 204 may couple the respective overlap elements 104. In some examples, the attachment features 204 are arranged and shaped to extend more longitudinally overall than circumferentially (e.g., as a plurality of longitudinally arranged elements attaching the overlapping portions 200), or may extend more perpendicularly or circumferentially overall, or may extend helically overall. The attachment features 204 may be formed or configured to resist the overlapping portions 200 from unfolding or detaching from one another. The attachment features 204, in some examples, include at least one adhesive region(s), filament(s), suture(s), tape(s), bonded region(s), soldered region(s), stitch(es), and/or any other mechanism suitable to couple the overlapping portions 200 with one another. The attachment features 204 may be positioned axially or longitudinally with respect to the body 102 of the implantable conduit 100.

In some examples, the attachment features 204 attach to an outer surface of the implantable conduit 100, and in other examples, the attachment features 204 attach to an inner surface of the implantable conduit 100. In some examples, the attachment features 204 may be elongate strips, which may include adhesive material to form adhesive strips or tapes, extending in a longitudinal direction along a length of the implantable conduit 100 to couple a first overlap element 104A to a second overlap element 104B. The attachment features 204 help retain each of the overlap elements 104 at a predetermined displacement with respect to its neighboring overlap elements 104 and to help maintain the overlap elements 104 orientation relative to each other. In some examples, the attachment features 204 include one or more stent elements instead of, or in addition to the other features such as adhesive strips or tapes. In some examples, the attachment features 204 are separated equidistally apart from each other (e.g., along the circumference and/or length of the implantable conduit 100). In some examples, one or more of the attachment features 204 are disposed relatively to one another at varying distances.

The attachment features 204 may be formed of a variety of materials and in a variety of configurations. The attachment features may be formed of metallic, polymeric, or any other suitable, biocompatible material functioning to help maintain the overlap elements 104 in their assembled positions. In some examples, the attachment features 204 include polymeric adhesives, such as fluoropolymers, epoxies, polyurethanes, acrylates, or others. The attachment features 204 may be formed of flexible material (e.g., compliant and/or elastic materials) to facilitate bending of the implantable conduit 100, for example.

In some examples, the attachment features 204 take the form of a flexible adhesive strips or tapes including one or more carrier layers and more or more adhesive layers or components. For example, the flexible adhesive strips may include a carrier layer of expanded polytetrafluoroethylene (ePTFE) and a coating of fluorinated ethylene propylene (FEP). In some examples, the adhesive material may be bonded using thermal treatments (e.g., as a thermoplastic or thermosetting material), or otherwise activated (e.g., UV-cured). The attachment features 204 may include bonded/adhered portions and unbonded/unadhered portions. In some examples, adhesive material is only present along predetermined portions of the attachment features 204 facilitating selective bonding and/or curing. In others, selective bonding processes are used (e.g., selective thermal bonding) to achieve selective bonding and/or curing.

The overlapping portions 200 and the attachment features 204 of the implantable conduit 100 combine to define apertures 206, which may alternatively be termed as openings, ports, or fenestrations, on the implantable conduit 100. In some examples, the plurality of overlap elements 104 define a plurality of adjacent pairs of overlap elements (e.g., overlap elements 104A and 104B) and a plurality of overlapping portions 200, each of the adjacent pairs of overlap elements being separable at a corresponding one of the plurality of overlapping portions 200 to open the aperture 206 that defines a path between the inner lumen 110 and the outer surface 112 of the implantable conduit 100.

In some examples, one of the overlapping portions 200 may only define one aperture 206 or a plurality of apertures 206 as desired. In the example shown in FIG. 2, each aperture 206 is defined by a first end 208A of the first overlap element 104A, and a second end 208B of the neighboring second overlap element 104B, and one or more of the attachment features 204. In some examples, the total number and arrangement of apertures 206 depends upon the number of attachment features 204 as well as the number of overlapping portions 200 in the implantable conduit 100. The apertures 206 are configured to be opened or fenestrated by selectively separating two adjacent overlap elements (e.g., the first overlap element 104A and the second overlap element 104B) apart from each other in a location between or intermediate to the attachment features 204. In some examples, markers may be placed at or proximate the apertures 206 to mark the locations of potential fenestrations for medical imaging.

In various examples, the implantable conduit 100 is configured such that the overlapping portions 200 have a closed configuration unless deformed or displaced to an open configuration. That is, under normal operating conditions, the overlapping portions 200 inhibit fluid or objects moving from inside the implantable conduit 100 to outside the implantable conduit 100, and vice versa. In some examples, the implantable conduit 100 is used within branched bodily conduits, such as the vasculature or the pancreatic, hepatic, or biliary ductal systems, of a patient. Also, the implantable conduit 100 may be configured to close the aperture(s) 206 in the overlapping portion(s) 200 upon removal of the external force separating the overlap elements 104 at the overlapping portion(s) 200, in turn sealing the path between the inner lumen 110 and the outer surface 112 of the implantable conduit 100.

Generally, internal and external pressures on the implantable conduit 100 vary according to a location in which the implantable conduit 100 is placed. In various examples, the overlapping portions 200 are configured (e.g., sized, shaped, and arranged) to remain sealed in response to pressure differentials across the wall or body 102 of the implantable conduit 100 such that the overlapping portions 200 of the implantable conduit 100 exhibit the closed configuration without requiring additional reinforcement, such as stent rings or other reinforcement, to maintain the overlapping portions 200 in a closed, or sealed configuration.

In some examples, one or more of the apertures is configured to be physically opened and fenestrated using a catheter, guidewire, or other fenestration tool (not shown). Regardless of mode of opening, in the absence of an external force the apertures 206 are configured to self-seal or return to the previous closed or sealed state in use, thus preventing flow therethrough.

In some examples, one or more of the apertures 206 may be opened or fenestrated in response to applying an external force on the implantable conduit 100. In such examples, the external force causes displacement on the overlap elements 104, which in turn causes creases to be formed in the overlapping portions 200. In some examples, where the overlap elements 104 are arranged in helical configuration, the external force may be a rotational force applied in a direction (clockwise or counterclockwise) opposite to the direction of the helical configuration (opposite a direction of winding) of the overlap elements 104. Regardless of mode of opening, in various examples in the absence of an external force the apertures 206 “self-seal” or return to the previous closed or sealed state after removal of an opening force, thus inhibiting fluid or objects passing therethrough.

In some examples, the implantable conduit 100 is made by forming the implantable conduit 100 by winding a sheet of membrane. The implantable conduit 100 includes a plurality of overlap elements 104 arranged in rows, and each overlap element 104 overlaps an adjacent row of overlap element 104. A plurality of attachment features 204 are attached to the plurality of overlap elements 104. The plurality of attachment features 204 are arranged essentially perpendicularly to the overlap elements 104 to maintain the plurality of overlap elements 104 relative orientation with each other. As shown, the implantable conduit 100 is configured to be fenestrated to form an aperture 206 at one or more locations along the implantable conduit 100 by selectively separating the overlap elements 104 from each other intermediate to the attachment features 204.

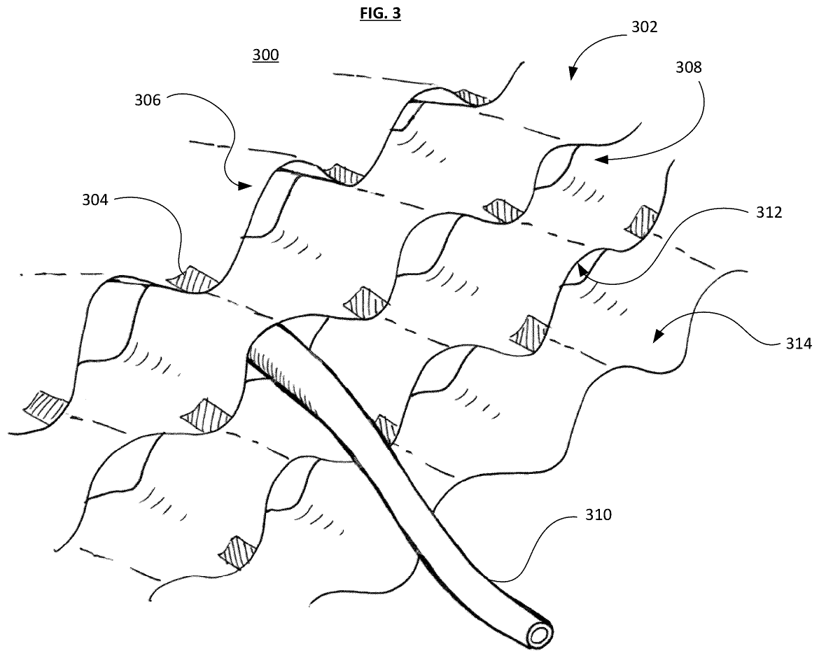

FIG. 3 shows a portion of an implantable conduit 300 with a plurality of overlap elements 302, where the implantable conduit 300 includes a plurality of attachment features 304 securing the overlap elements 302, according to some embodiments. As shown, the attachment features 304 are positioned at circumferentially staggered positions from one another about a circumference of the implantable conduit 300. The attachment features 304 may couple a pair of the overlap elements 302 being circumferentially offset not only from one another, but also from the attachment features coupling a neighboring, or adjacent pair of the overlap element 302. In this manner, the attachment features 304 of one pair of overlap elements 302 do not align longitudinally with the attachment features 304 of the next neighboring pair of overlap elements 302. For reference, a “pair” of overlap elements as used herein is meant to indicate two overlap elements that are coupled and define an overlapping portion, such as the overlapping portions 200 (FIG. 1). It should be understood that a single one of the overlap elements 302 may form more than one pair. In particular, as shown, a single one of the overlap elements 302 forms a “pair” with two different overlap elements 302 (two longitudinally adjacent ones) as shown in FIG. 3, for example.

In FIG. 3, for simplicity the attachment features 304 are shown as shaded areas, which may not actually be visible. The shaded areas may be understood to represent the relative locations of the attachment features 304 between the overlap elements 302. The attachment features 304 may be a layer of adhesive or a thermally bonded region, for example, or some other feature coupling the overlap elements 302. For example, the layer of adhesive may be a coating of polymeric material such as fluorinated ethylene propylene (FEP) or any other suitable bonding agent. Regardless, the attachment features 304 may be located between the overlap elements 302 and be hidden from view in FIG. 3. The attachment features 304 generally act to hold the pairs of overlap elements 302 together. As previously referenced, according to some embodiments an external force applied to the implantable conduit 300, such as a mechanical force pressing into one or more of the apertures 308 to open the one or more apertures 308, such as a mechanical or insertion force from a catheter or guidewire. In other examples, an external force is applied by imparting a twisting or torsional force on the implantable conduit 300 to cause creases 306 around the zones containing the attachment features 304 such that the overlap elements 302 deflect around the one or more apertures 308. As shown, a torsional force may cause the pairs of overlap elements 302 to crease inwardly and outwardly around the apertures 308 as shown in FIG. 3 as a result of such external force.

Regardless of the opening, or expansion mechanism used to open the one or more apertures 308, in some examples, a secondary device structure 310, which may be another implantable conduit (e.g., a catheter, a guidewire, graft, stent graft) or some other device, is passed or received through the aperture 308 (from outside the implantable conduit 300 to inside the implantable conduit 300 or vice versa as appropriate). The secondary device structure 310 may form a branch, or additional flow lumen extending from the lumen of the implantable conduit 300. When used as a vascular implant, for example, the main implantable conduit 300 may perfuse a main vessel while the secondary device structure 310, which may be another implantable conduit, may perfuse a side vessel smaller than the main vessel.

Furthermore, each of the overlap elements 302 has an inward facing surface 312 and an outward facing surface 314 such that two neighboring or adjacent pairs of overlap elements 302 are positioned to define the overlapping portion (for example, the overlapping portion 200 in FIG. 2) between the inward facing surface 312 of the first overlap element and the outward facing surface 314 of the second overlap element. The plurality of attachment features 304 couple the first overlap element to the second overlap element. Also, the inward facing surface 312 of the first overlap element and the outward facing surface 314 of the second overlap element are separable at the overlapping portion by an external force to open the one or more apertures 308 in the overlapping portion between at least two of the plurality of attachment features 304 such that the aperture(s) 308 defines a path between the inner lumen and outer surface of the implantable conduit, the path being sealed absent the external force.

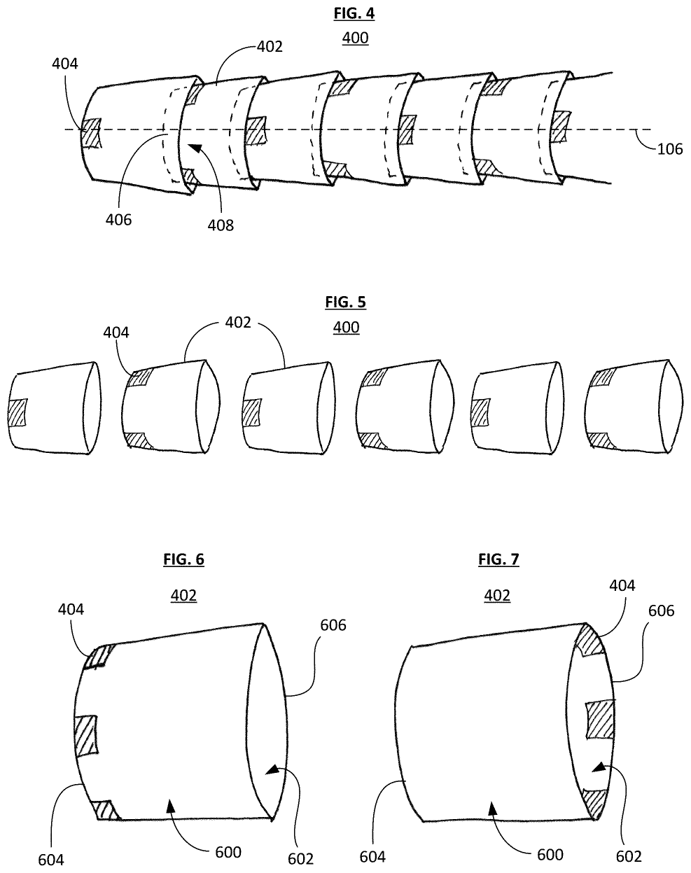

FIGS. 4 and 5 show another implantable conduit 400 configured with a plurality of overlap elements 402 that are separate and distinct tubular members, where the implantable conduit 400 includes has a plurality of attachment features 404 coupling the overlap elements 402, according to some embodiments. As shown, the overlap elements 402 form pairs of overlap elements 402 that share an overlapping portion 406 therebetween, which defines an aperture 408 that is openable in response to an external force. The overlap elements 402, unlike the overlap elements 104 in FIGS. 1 and 2, are separate, tubular elements (e.g., rather than being continuously formed) that are aligned along the longitudinal axis 106 to form the implantable conduit 400. There can be any number of attachment features 404 coupling the overlap elements 402 and may be configured and positioned in a variety of manners, including any of those associated with the attachment features 204 and 304 previously described.

FIGS. 6 and 7 show examples of separate and distinct overlap elements 402 that can be used to form the implantable conduit 400 according to some embodiments. FIG. 6 shows attachment features 404 disposed on a first end 604 of an exterior surface 600 of one of the overlap elements 402, whereas FIG. 7 shows attachment features 404 disposed on a second end 606, which is opposite the first end 604, and on an interior surface 602 of one of the overlap elements 402.

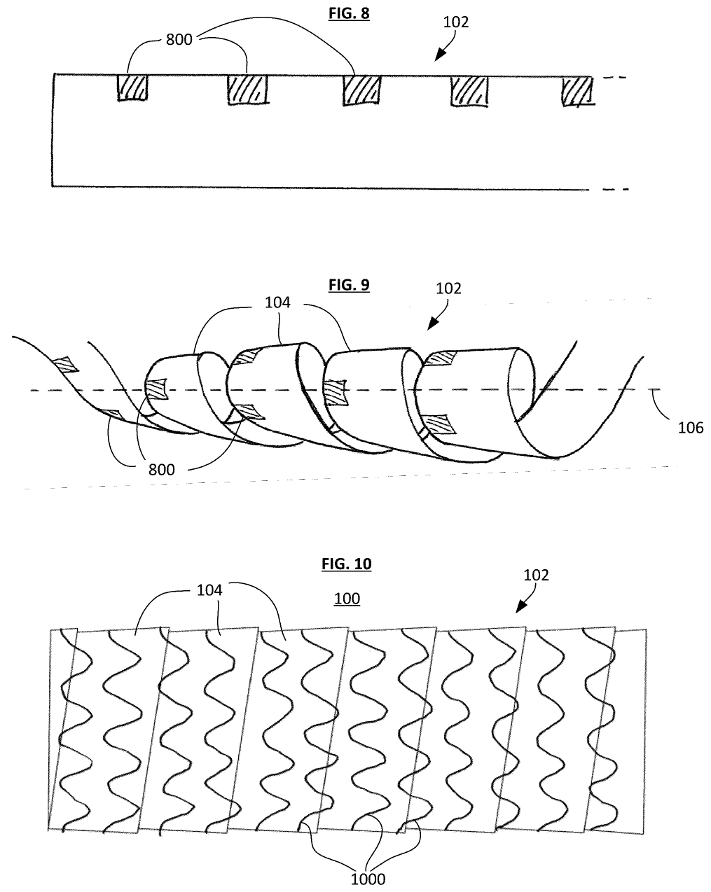

FIG. 8 shows another example in which the wall or body 102 of the implantable conduit 100 is formed from an element that is wrapped helically to form the plurality of overlap elements 104, such as the example of FIGS. 1 and 2. As shown, a plurality of attachment features 800 are located along an edge of the covering 102 at a desired spacing (e.g., an equidistant spacing) such that when the covering 102 is wound to form the helical configuration as shown in FIG. 9, the attachment features 800 have a staggered configuration along the longitudinal axis 106 of the implantable conduit 100. The attachment features 800 may be formed in a variety of manners, including methods in which they are pre-formed on the wall or body 102 (e.g., as an adhesive layer) or formed along the wall or body 102 subsequent to helically formation (e.g., via thermal bonding), for example.

FIG. 10 shows the implantable conduit 100 with a plurality of stent elements 1000, also described as support members 1000, coupled to the plurality of overlap elements 104. The stent elements 1000 can be made of biocompatible metallic or polymeric materials as desired, such as stainless steel or nitinol, among others. The support members 1000 may take on an undulating pattern, or a variety of configurations as desired, such as a serpentine configuration as shown in FIG. 10. In some examples, a plurality of the stent elements 1000 are formed using a single elongate member (e.g., wire) having a serpentine or undulating arrangement and which is helically wound around a circumference of the implantable conduit 100 and attached to the wall 102 or body 102. In such examples, each of the stent elements 1000 may be defined by a single turn of the elongate member. In some examples, one or more of the stent elements 1000 is formed of a separate annular of wire (e.g., as a ring-shaped element). The stent elements 1000 can be attached to an exterior surface or an interior surface of the covering 102 (e.g., using adhesives, tapes, fastening elements, such as sutures, or combinations thereof).

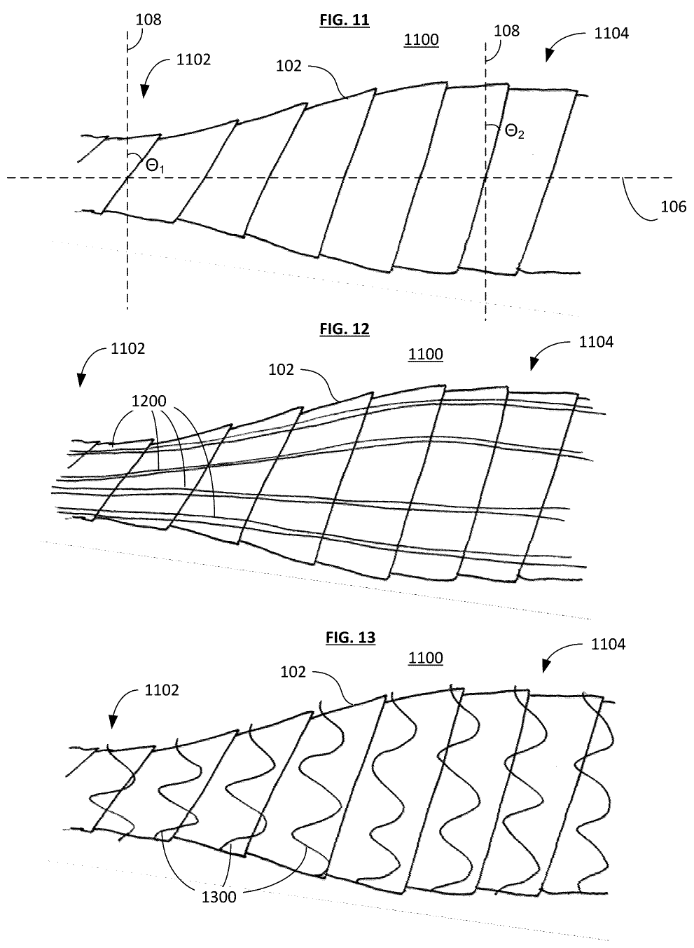

FIGS. 11 to 13 show additional implantable conduit variations. As shown in FIG. 11, the implantable conduit 1100 may have a tapering, or tapered configuration including a narrow portion 1102 or smaller diameter portion 1102 and a wider portion 1104 or greater diameter portion 1104 that has greater diameter than the narrower portion 1102. The difference in diameters may be a smooth transition a stepped transition, and there may be a single taper or multiple tapers as desired. In some examples, the tapered diameter can be achieved by varying the wrapping angle of the body or wall 102 in forming the implantable conduit 1100. For example, the narrower portion 1102 may have a first wrapping angle Θ₁ that is greater than a second wrapping angle Θ₂ of the wider portion 1104, where each wrapping angle is measured with respect to an axis 108 perpendicular to the longitudinal axis 106.

In some examples, the first wrapping angle Θ₁ may greater than the second wrapping angle Θ₂ by from about 10 degrees to about 20 degrees, about 20 degrees to about 30 degrees, about 30 degrees to about 40 degrees, or any range therebetween. FIG. 12 shows another various including a plurality of attachment features 1200 extending longitudinally along the implantable conduit 1100 at various circumferential positions about the circumference of the implantable conduit 1100. The attachment features 1200 may extend along an interior surface, an exterior surface, or within layers of the implantable conduit 1100 as desired. The attachment features 1200 may curve to match a contour of the implantable conduit 1100. FIG. 13 shows another variation in which a plurality of stent elements 1300 are attached to the implantable conduit 1100. The attachment features 1200 and the stent elements 1300 may take any of the configurations described in associations with any of the prior examples of implantable conduits 100, 300 and 400.

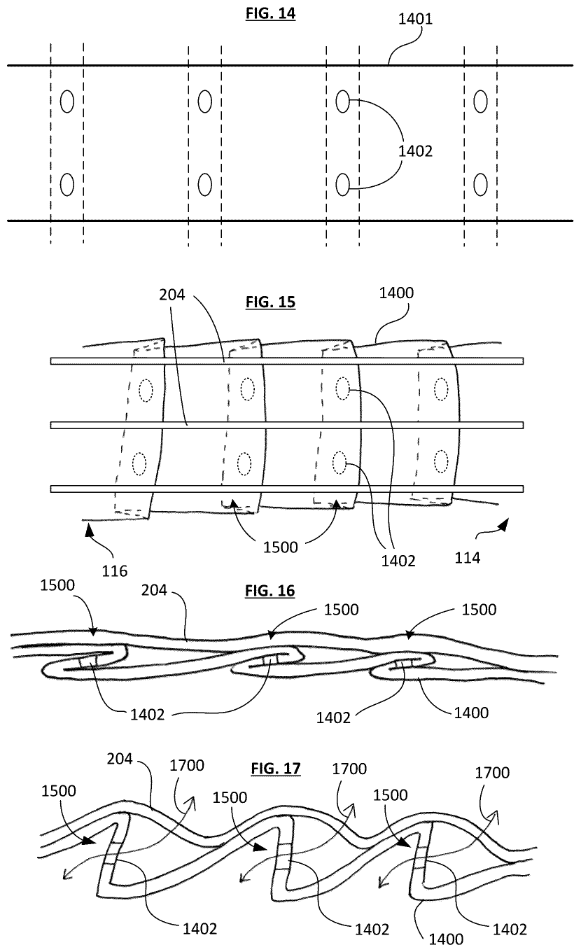

FIGS. 14 to 17 show additional implantable conduit variations. As shown in FIG. 14, a tube 1401 of flexible material may be used to form an implantable conduit 1400, according to some embodiments. As shown, the tube 1401 of flexible material (e.g., such as the materials previously described for the body or wall 102) may be fenestrated with a plurality of apertures 1402, which may also be referred to as openings or fenestrations, at desired intervals along the length of the implantable conduit 1400. Each of the apertures 1402 is longitudinally aligned with some of the other apertures 1402 and also axially aligned with some of the other apertures 1402. There may be any number of apertures 1402 on the implantable conduit 1400, in any of a number of desired configurations. One or more of the apertures 1402 may be aligned essentially circumferentially about and/or axially along the implantable conduit 1400 as desired.

The tube 1401 may be folded (e.g., along the transverse, broken lines shown) to define a plurality of overlap elements 1500 in the form of multiple pleats (as such, the overlap elements 1500 may also be referred to as pleats when folded as shown) that at least partially overlap along a length of the implantable conduit 1400 between the first end 114 and the second end 116 as shown in FIG. 15. In some examples, the tube 1401 is fenestrated within a pleat to provide a side portal along the length of the tube 1401, the side portal being the aperture 1402. Such fenestrations or apertures 1402 allow for the introduction of a side branch tube (e.g., secondary device structure 310 in FIG. 3) therethrough. The plurality of overlap elements 1500 are defined by a tubular body having a pleated configuration to define a plurality of pleats, where the plurality of attachment features 204 couple the overlap elements 1500 in the pleated configuration. Though the pleats may be circumferentially oriented with the aperture 1402 formed in one of the pleats in some examples, it should be understood that longitudinally oriented pleats may similarly be employed. As shown, attachment features 204, or any of those previously described, are utilized to couple the overlap elements 1500 in the overlapped configuration.

In some examples, the attachment features 204 are longitudinal elements or stiffening elements, which may include one or more of at least one spine, tape, and/or stent element, for example, to reinforce the pleats so as to resist the pleats from unfolding upon application of tension along the length of the tube 1401. In other examples, the overlap elements are formed to maintain the overlapped configuration (e.g., through heat setting or other treatments). As shown in FIG. 16, the overlap elements 1500 are in the closed configuration, forming pleats, with the apertures 1402 formed in the pleats and closed to inhibit fluid or objects passing between the interior and exterior of the implantable conduit 1400.

As shown in FIG. 17, when an external force is applied as previously disclosed (e.g., via being physically opened and fenestrated using a catheter, guidewire, or other fenestration tool), the implantable conduit 1400 becomes distorted and the distortion causes the overlap elements 1500 to become partially unpleated, thereby allowing the apertures 1402 to be in an open or unsealed configuration. In the open or unsealed configuration, solids or fluids are allowed to pass through the apertures 1402 as shown by the arrows 1700 in FIG. 17.

In view of the foregoing, the implantable conduit 1400 may be configured to be fenestrated through the apertures 1402 within the overlap elements 1500 when the overlap elements 1500 become partially unpleated. In some examples, the fenestrations or apertures 1402 may be operated to an open configuration to provide a side portal along the length of the implantable conduit 1400 to allow for a secondary device, such as the side branch tube, to be passed through the side portal. In some examples, the overlap elements 1500, or pleats, may be configured to overlap and resist leakage around the secondary device (not shown), which may be the side branch tube.

In some examples, the attachment features 204, or other versions previously described, may act to allow the distortion of the overlap elements 1500 to open the apertures 1402, while also providing sufficient resistance to such deformation to return to a closed configuration and/or seal around a secondary device passed through one or more of the apertures 1402. In some examples, the locations of the apertures 1402, or points of fenestration, are indicated by one or more radiopaque markers placed at or near the locations on the tube 1401. Attachment features and/or support elements such as those previously described may be incorporated to help that reinforce the overlap elements 1500 to resist unwanted unfolding (e.g., upon application of tension along the length of the implantable conduit 1400).

It should be understood that, although the attachment features 204 are shown to be attached to an exterior surface of the implantable conduit 1400, other examples may have the attachment features 204 attached to an interior surface of the implantable conduit 1400. Furthermore, instead of the attachment features 204 that extend along the length of the implantable conduit 1400, some examples may employ a plurality of shorter attachment features as appropriate. Also, these shorter attachment features may be positioned in a staggered configuration as previously shown in FIG. 3 for example.

In some examples, an implantable conduit 1400 is made by forming an aperture 1402 at one or more locations of the implantable conduit 1400 comprising a sheet of membrane. The implantable conduit 1400 is folded to form a plurality of overlap elements or pleats 1500. The aperture 1402 is at the one or more locations within the pleats 1500. A plurality of attachment features 204 are attached to the folded implantable conduit 1400. The attachment features 204 are arranged essentially perpendicularly to the pleats 1500 to maintain the pleats 1500 against a surface of the implantable conduit 1400 to keep the aperture 1402 closed. The aperture 1402 at the one or more locations within the pleats 1500 is configured to be opened in response to selectively and at least partially unpleating the pleats 1500 from the surface of the implantable conduit 1400 intermediate to the attachment features 204.

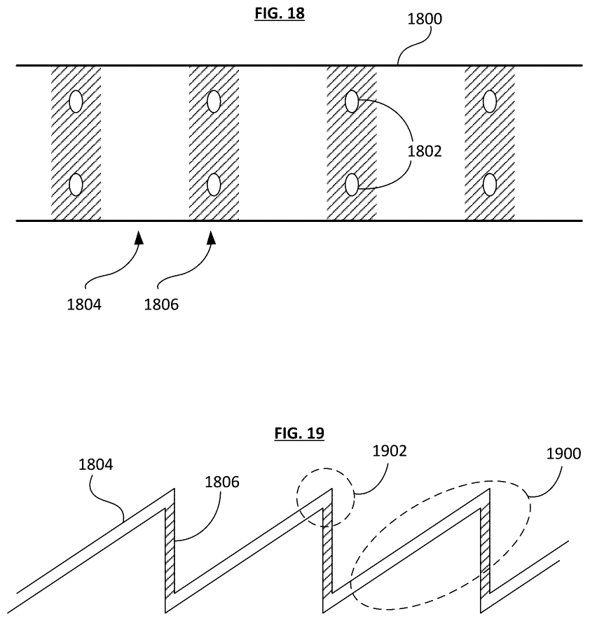

FIGS. 18 and 19 show additional implantable conduit features that may be implemented in addition to or as an alternative in any of the foregoing example designs. As shown in FIG. 18, a tube 1800 of flexible material may have alternating sections of flex portions 1804 and stiffer portions 1806, according to some embodiments. The flex portions 1804 are relatively more flexible (e.g., easier to bend, move, or otherwise deform) than the stiffer portions 1806. The stiffer portions 1806 may be formed by any suitable means, such as applying additional layer(s) of material, for example non-compliant polymeric material, plastic material, or adhesive material that stiffens when dried, to name a few. In some examples, the stiffer portions 1806 may be stiffened by altering the physical or chemical properties of the flex portions 1804 such as by curing, sintering, or tempering of the flex portions 1804, among other methods. Therefore, in these cases, the flex portions 1804 and the stiffer portions 1806 are made of the same material. Additionally, the stiffer portions 1806 may have one or more apertures 1802 (also referred to as openings or fenestrations) to let fluid flow therethrough at desired intervals along the length of the implantable conduit 1800.

The tube 1800 may be folded to form multiple pleats 1900 by at least partially overlapping the stiffer portions 1806 onto the flex portions 1804. FIG. 19 shows the tube 1800 being folded, such that each fold 1902 is located between the stiffer portion 1806 and the flex portion 1804, so when the tube 1800 is completely folded, the stiffer portions 1806 are at least partially hidden underneath the neighboring flex portions 1804. In these embodiments, the stiffer portions 1806 are stiff enough to resist longitudinal compression and/or not compress longitudinally, thereby maintaining the pleated configuration of the folded tube 1800. In some examples, the pleated configuration is maintained without using any attachment features (e.g., 204 shown in FIG. 15) to prevent the folded tube 1800 from unfolding.

Inventive concepts of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable conduit that is tubular in shape and has an inner lumen and an outer surface, the implantable conduit comprising:
a plurality of overlap elements including a first overlap element and a second overlap element positioned adjacent the first overlap element to define an adjacent pair of the plurality of overlap elements, the first overlap element overlapping onto the second overlap element to define an overlapping portion between an inward facing surface of the first overlap element and an outward facing surface of the second overlap element; and
a plurality of attachment features coupling the first overlap element to the second overlap element, the inward facing surface of the first overlap element and the outward facing surface of the second overlap element being separable at the overlapping portion by an external force to open an aperture in the overlapping portion between at least two of the plurality of attachment features such that the aperture defines a path between the inner lumen and outer surface of the implantable conduit, the path being sealed absent the external force.

2. The implantable conduit of claim 1, wherein the plurality of overlap elements are arranged along a length of the implantable conduit and the overlapping portion extends in a circumferential direction.

3. The implantable conduit of claim 1, wherein the plurality of overlap elements define a plurality of adjacent pairs of overlap elements and a plurality of overlapping portions, each of the adjacent pairs of overlap elements being separable at a corresponding one of the plurality of overlapping portions to open an aperture that defines a path between the inner lumen and the outer surface of the implantable conduit.

4. The implantable conduit of claim 1, wherein the plurality of attachment features are elongate strips of material extending in a longitudinal direction along a length of the implantable conduit to couple the first overlap element to the second overlap element.

5. The implantable conduit of claim 1, wherein the implantable conduit is configured to close the aperture in the overlapping portion upon removal of the external force separating the overlap elements at the overlapping portion to seal the path between the inner lumen and outer surface of the implantable conduit.

6. The implantable conduit of claim 1, wherein the attachment features include adhesive material.

7. The implantable conduit of claim 1, wherein the attachment features are positioned at circumferentially staggered positions from one another about a circumference of the implantable conduit.

8. The implantable conduit of claim 1, further comprising a secondary device structure received through the aperture.

9. The implantable conduit of claim 1, wherein the implantable conduit defines a varying diameter.

10. The implantable conduit of claim 1, further comprising a plurality of stent elements coupled to the plurality of overlap elements.

11. The implantable conduit of claim 1, wherein the plurality of overlap elements are defined by a continuous, helical length of material overlapped edgewise onto itself; or wherein the plurality of overlap elements include discrete, adjacent circumferential rings of material that are overlapped in an edgewise manner.

12. The implantable conduit of claim 1, wherein the plurality of overlap elements are defined by a tubular body having a pleated configuration to define a plurality of pleats, the plurality of attachment features coupling the overlap elements in the pleated configuration.

13. The implantable conduit of claim 12, wherein the pleats are circumferentially oriented; and optionally, wherein the aperture is formed in one of the pleats.

14. An implantable conduit comprising:
a tube of flexible material having a length, a first end, and a second end, the tube formed with multiple pleats along its length;
a stiffening element reinforcing the pleats so as to resist the pleats from unfolding upon application of tension along the length of the tube;
wherein the tube is configured to be fenestrated within a pleat to provide a side portal along the length of the tube to allow for the introduction of a side branch tube through the portal; and
wherein the pleat is configured to overlap and resist leakage around the side branch tube.

15. The implantable conduit of claim 14, wherein the tube includes a radiopaque marker to indicate a point of fenestration.

16. The implantable conduit of claim 14, wherein the stiffening element comprises a stent element.

17. The implantable conduit of claim 14, wherein the stiffening element comprises a tape.

18. An implantable conduit comprising:
a tube of flexible material having a length, a first end, and a second end, the tube formed with multiple pleats along its length;
a longitudinal element attached along the length to resist the pleats from unfolding upon application of tension along the length of the tube;
wherein the tube is configured to be fenestrated within a pleat to provide a side portal along the length of the tube to allow for the introduction of a side branch tube through the portal; and
wherein the pleat is configured to overlap and resist leakage around the side branch tube.

19. The implantable conduit of claim 18, wherein the longitudinal element comprises at least one spine.

20. The implantable conduit of claim 18, wherein the longitudinal element comprises at least one tape.
